(19) United States
(12) Patent Application Publication
(10) Pub. No.: US 2022/0265477 A1
(43) Pub. Date: Aug. 25, 2022

(54) Hand Dressing For Use With Negative Pressure Wound Therapy, Negative Pressure Wound Therapy System Comprising The Same, Method For Controlling A Pump Coupled To Said Dressing

(71) Applicant: KCI Licensing, Inc., San Antonio, TX (US)

(72) Inventors: Christopher Brian LOCKE, Bournemouth (GB); James Killingworth SEDDON, Wimborne (GB); Thomas Alan EDWARDS, Hampshire (GB)

(21) Appl. No.: 17/630,842

(22) PCT Filed: Jul. 30, 2020
(86) PCT No.: PCT/IB2020/057194

§ 371 Date: Jan. 27, 2022

Related U.S. Application Data
(60) Provisional application No. 62/883,424, filed on Aug. 6, 2019.

(51) Int. Cl. A61F 13/00; A61F 13/02; A61F 13/10; A61M 1/00

(52) U.S. Cl. CPC A61F 13/00068; A61F 13/0216; A61F 13/104; A61M 1/73; A61M 1/74; A61M 1/917; A61M 2205/0283; A61M 2205/056; A61M 2205/332; A61M 2205/3324

(57) ABSTRACT

A dressing includes a first manifold layer, a second manifold layer, a first barrier layer coupled to the first manifold layer, a second barrier layer coupled to the second manifold layer, a first fenestrated film layer coupled to the first manifold layer, and a second fenestrated film layer coupled to the second manifold layer. The first manifold layer and the second manifold layer are positioned between the first barrier layer and the second barrier layer. The first fenestrated film layer and the second fenestrated film layer are positioned between the first manifold layer and the second manifold layer.

### CROSS-REFERENCE TO RELATED APPLICATIONS

This application claims the benefit of priority to U.S. Provisional Application No. 62/883,424, filed on Aug. 6, 2019, which is incorporated herein by reference in its entirety.

### BACKGROUND

The present invention relates generally to the field of treating wounds (e.g., burns, lacerations, surgical incisions, sores, ulcers, damaged tissue, nerve damage, etc.) and more particularly to negative pressure wound therapy systems with instillation therapy. Negative pressure wound therapy refers to the application of negative pressure (relative to atmospheric pressure) to a wound bed to facilitate healing of the wound bed. Negative pressure may be applied in coordination with instillation therapy, in which instillation fluid (e.g., cleansing fluid, medicated fluid, antibiotic fluid, irrigation fluid) is applied to the wound bed. Negative pressure and instillation wound therapy (NPWTi) may facilitate removal of wound exudate and other debris from the wound bed and otherwise support healing.

One common location for a wound (e.g., a burn) that could benefit from NPWTi is on a patient's hand. However, standard NPWTi dressings may be challenging to use on a hand due to the shape, size, contours, articulation, etc. of a hand. Accordingly, hand-specific dressings may facilitate improved NPWTi for hand wounds.

### SUMMARY

One implementation of the present disclosure is a dressing. The dressing includes a first manifold layer, a second manifold layer, a first barrier layer coupled to the first manifold layer, and a second barrier layer coupled to the second manifold layer. The first manifold layer and the second manifold layer are positioned between the first barrier layer and the second barrier layer. The first manifold layer, the second manifold layer, the first barrier layer, and the second barrier layer are hand-shaped. The first barrier layer is coupled to the second barrier layer along a hand portion of a perimeter of the dressing and separated from second barrier layer along a wrist portion of the perimeter of the dressing.

In some embodiments, the dressing includes a first fenestrated film layer coupled to the first manifold layer and a second fenestrated film layer coupled to the second manifold layer. The first fenestrated film layer and the second fenestrated film layer are positioned between the first manifold layer and the second manifold layer. The dressing may also include an anchor weld extending along a subportion of the hand portion of the perimeter of the dressing. The anchor weld is coupled to the first barrier layer, first manifold layer, first fenestrated film layer, second fenestrated film layer, second manifold layer, and second barrier layer and configured to restrict relative motion therebetween.

In some embodiments, the dressing is configured to receive a hand of a patient between the first fenestrated film layer and the second fenestrated film layer. The dressing may include an adhesive cuff positioned at the wrist portion of the perimeter of the dressing. The adhesive cuff is configured to be coupled to a wrist of a patient. The first barrier layer and the second barrier layer may be substantially impermeable to air, and the adhesive cuff may be configured to provide a substantially airtight seal between the first and second barrier layers and the wrist of the patient.

In some embodiments, a connection pad is coupled to the first barrier layer and configured to couple the dressing to a tube coupled to a pump such that the pump is in pneumatic communication with the first manifold layer and the second manifold layer. The pump may be controllable to selectively establish a first level of negative pressure at the dressing and a second level of negative pressure at the dressing. The dressing may be configured to substantially prevent bending of the dressing when the first level of negative pressure is established at the dressing and to allow bending of the dressing when the second level of negative pressure is established at the dressing. The second level of negative pressure corresponds to a physiotherapy mode.

In some embodiments, the dressing includes one or more sensors coupled to the dressing. The one or more sensors include one or more of a moisture sensor, a humidity sensor, a pH sensor, and a strain sensor. The one or more sensors can transmit measurements via a wireless network.

In some embodiments, the first barrier layer includes a plurality of knuckle flexion points. Each knuckle flexion point is configured to facilitate bending of the first barrier layer at the knuckle flexion point.

In some embodiments, the thicknesses of the first manifold layer and the second manifold layer are less than approximately 10 millimeters.

In some embodiments, the first fenestrated film layer is positioned between the first manifold layer and tissue of a patient when the dressing is applied to the patient. When the dressing is applied to a patient, the first fenestrated film layer may prevent the first manifold layer from contacting a tissue of the patient and the second fenestrated film layer prevents the second manifold layer from contacting the tissue of the patient.

In some embodiments, the first fenestrated film layer includes a polyurethane film. The first fenestrated film layer may include a non-porous polyurethane film. A thickness of the first fenestrated film layer is approximately thirty microns. A thickness of the first fenestrated film layer may be less than a thickness of the first barrier layer. The first fenestrated film layer may include a plurality of fenestrations having lengths of approximately three millimeters. The fenestrated film layer includes a plurality of fenestrations, the plurality of fenestrations arranged in a plurality of staggered rows.

Another implementation of the present disclosure is a negative pressure wound therapy system. The system includes a glove-shaped dressing, a strain sensor coupled to the glove-shaped dressing and configured to collect a measurement of a strain of the glove-shaped dressing, a tube coupled to the glove-shaped dressing, a pump coupled to the tube, and a controller configured to receive the measurement from the strain sensor and to control the pump to establish a negative pressure at the glove-shaped dressing.

In some embodiments, the glove-shaped dressing is configured to be sealed around a hand of a patient. The glove-shaped dressing can restrict bending of the hand when a first level of negative pressure is established at the glove-shaped dressing by the pump.

In some embodiments, the controller is configured to determine whether the measurement from the strain sensor exceeds a threshold value, and, in response to a determination that the measurement from the strain sensor exceeds a threshold value, control the pump to provide a second level of negative pressure at the glove-shaped dressing. The second level is closer to ambient air pressure than the first level. The glove-shaped dressing may allow bending of the hand when the second level of negative pressure is established at the glove-shaped dressing by the pump.

In some embodiments, the strain sensor transmits the measurement to the controller via a wireless network. The strain sensor may include an electroactive polymer strain sensor. The strain sensor may extend from a wrist region of the glove-shaped dressing to a fingertip region of the hand shaped dressing.

In some embodiments, the system includes a humidity sensor and a moisture sensor. The controller is configured to receive measurements from the humidity sensor and the moisture sensor via a wireless network.

Another implementation of the present disclosure is a method for controlling a pump coupled to a glove-shaped dressing. The method includes controlling the pump to establish a first level of negative pressure at the glove-shaped dressing, receiving a measurement of a strain on the glove-shaped dressing from a strain sensor coupled to the glove-shaped dressing, determining whether the measurement of the strain exceeds a threshold value, and, in response to a determination that the measurement of the strain exceeds the threshold value, initiating a physiotherapy mode by controlling the pump to establish a second level of negative pressure at the glove-shaped dressing. The second level of negative pressure is closer to ambient air pressure that the first level of negative pressure.

In some embodiments, the method includes determining that the measurement of the strain on the glove-shaped dressing is below the threshold value for more than a threshold duration, and, in response to a determination that the measurement of the strain on the glove-shaped dressing returned below the threshold value for more than the threshold duration, exiting the physiotherapy mode by controlling the pump to establish the first level of negative pressure at the glove-shaped dressing.

Another implementation of the present disclosure is a method of treating a wound on a hand of a patient, the hand extending from a wrist. The method includes substantially enclosing the hand in a fenestrated film, inserting the hand and the fenestrated film into a glove via a wrist opening of the glove. The glove includes a manifolding layer and a barrier layer. The method includes sealing the wrist opening around the wrist of the patient, coupling the glove to a tube, coupling the tube to a pump, and operating the pump to establish a negative pressure at the hand.

In some embodiments, substantially enclosing the hand in the fenestrated film includes applying a first portion of the fenestrated film to a first side of the hand, applying a second portion of the fenestrated film to a second side of the hand, and mating a sub-portion of the first portion of the fenestrated film to a sub-portion of the second portion of the fenestrated film. The first portion is configured to adhere to the second portion.

In some embodiments, coupling the glove to the tube includes cutting a hole in the barrier layer and coupling a connection pad to the barrier over the hole. The connection pad is coupled to the tube.

Another implementation of the present disclosure is a dressing. The dressing includes a first manifold layer having a central region and five peninsular projections extending therefrom in the shape of a hand, a second manifold layer having a central region and five peninsular projections extending therefrom in the shape of a hand, a first barrier layer having a central region and five peninsular projections extending therefrom in the shape of a hand, and a second barrier layer having a central region and five peninsular projections extending therefrom in the shape of a hand. The first barrier layer is adjacent to the first manifold layer and the second barrier layer is adjacent to the second manifold layer. The first manifold layer and the second manifold layer are positioned between the first barrier layer and the second barrier layer to form a layered glove-shaped assembly. The first barrier layer is sealed to the second barrier layer along the five peninsular projections and sides of the central region and separated from second barrier layer along a bottom of the central region.

In some embodiments, the dressing includes a first fenestrated film layer having a central region and five peninsular projections extending therefrom in the shape of a hand and a second fenestrated film layer having a central region and five peninsular projections extending therefrom in the shape of a hand. The first fenestrated film layer is adjacent to the first manifold layer and the first fenestrated film layer is adjacent to the second manifold layer. The first fenestrated film layer and the second fenestrated film layer are positioned between the first manifold layer and the second manifold layer.

In some embodiments, the dressing includes an adhesive cuff positioned at the bottom of the central region.

In some embodiments, the dressing includes one or more sensors coupled to the dressing. The one or more sensors include one or more of a moisture sensor, a humidity sensor, a pH sensor, or a strain sensor. The one or more sensors are configured to transmit measurements via a wireless network.

Another implementation of the present disclosure is a dressing. The dressing includes an enclosure configured to enclose an appendage. The enclosure includes a film layer that includes a plurality of fenestrations configured to expand in response to a pressure gradient across the film layer. The enclosure also includes a barrier layer and a manifold layer disposed between the film layer and the barrier layer. The film layer is configured to contact the appendage.

In some embodiments, the dressing includes a second barrier layer coupled to the barrier layer along a portion of a perimeter of the first barrier layer. The dressing may also include a second film layer that includes a second plurality of fenestrations configured to expand in response to a pressure gradient across the second film layer. The dressing may also include second manifold layer disposed between the second film layer and the second barrier layer. The second film layer may be configured to contact the appendage.

In some embodiments, when the dressing is applied to the appendage, the film layer prevents the manifold layer from contacting the appendage. In some embodiments, the film layer comprises a polyurethane film. The film layer may include a non-porous polyurethane film. A thickness of the film layer may be approximately thirty microns and/or may be less than a thickness of the barrier layer. The film layer may include a plurality of fenestrations having lengths of approximately three millimeters. The film layer may include a plurality of fenestrations arranged in staggered rows.

### BRIEF DESCRIPTION OF THE DRAWINGS

FIG. 1 is a perspective view of a negative pressure and instillation wound therapy (NPWTi) system, according to an exemplary embodiment.

FIG. 2 is a block diagram of the NPWTi system of FIG. 1, according to an exemplary embodiment.

FIG. 3 is a top view of dressing for treating a hand wound and for use with the NPWTi system of FIGS. 1-2, according to an exemplary embodiment.

FIG. 4 is a first cross-section view of the dressing of FIG. 3, according to an exemplary embodiment.

FIG. 5 is a second cross-section view of the dressing of FIG. 3, according to an exemplary embodiment.

FIG. 6 is a third cross-section view of the dressing of FIG. 3, according to an exemplary embodiment.

FIG. 7 is an illustration of a wound-dressing interface for use with a glove-shaped dressing used with the NPWTi system of FIGS. 1-2, according to an exemplary embodiment.

FIG. 8 is a cross-section view of the wound-dressing interface of FIG. 7, according to an exemplary embodiment.

FIG. 9 is a cross-section view of the glove-shape dressing for use with the wound-dressing interface of FIG. 7, according to an exemplary embodiment.

FIG. 10 is a flowchart of a process for providing a physiotherapy mode with the NPWTi system of FIGS. 1-2, according to an exemplary embodiment.

### DETAILED DESCRIPTION

### Negative Pressure and Instillation Wound Therapy System

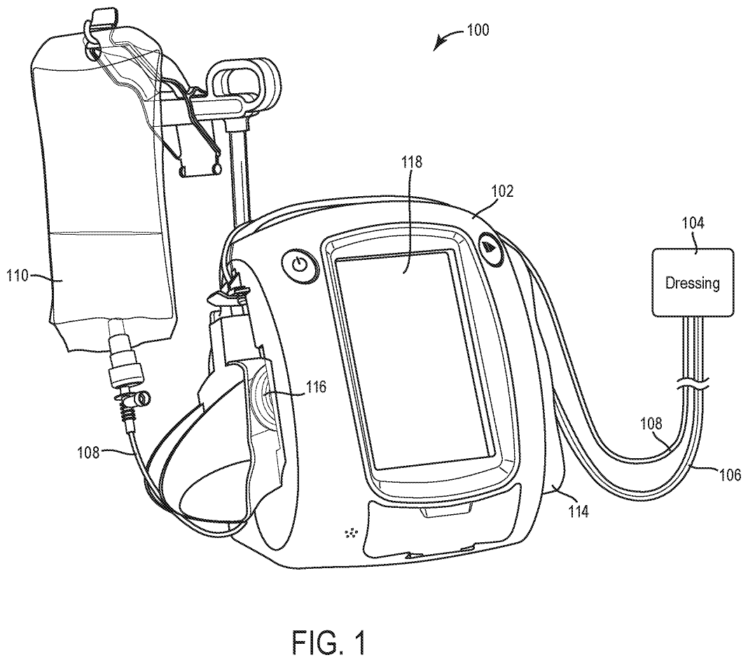

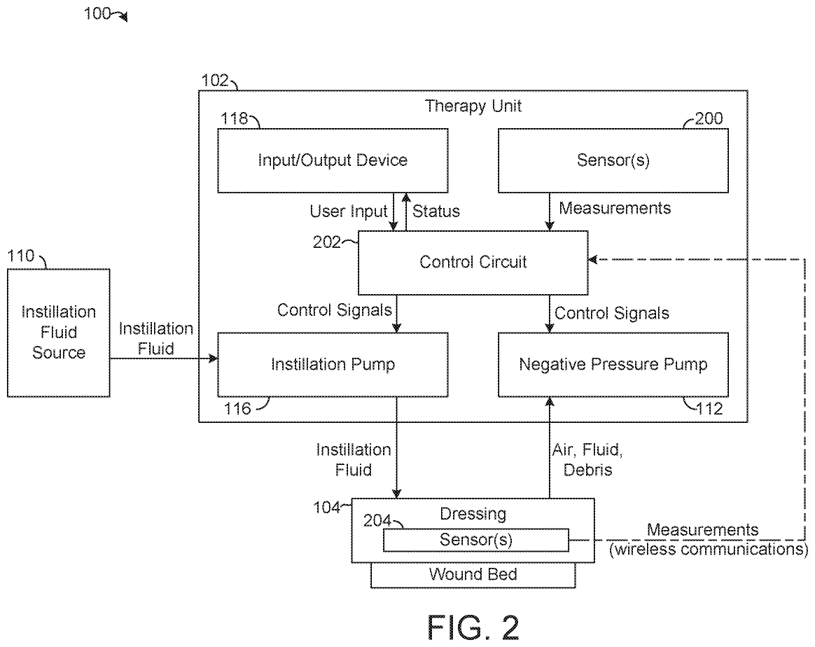

Referring to FIGS. 1 and 2, a negative pressure and instillation wound therapy (NPWTi) system 100 is shown, according to exemplary embodiments. FIG. 1 shows a perspective view of the NPWTi system 100, according to an exemplary embodiment. FIG. 2 shows a block diagram of the NPWTi system 100, according to an exemplary embodiment. The NPWTi system 100 is shown to include a therapy unit 102 fluidly coupled to a dressing 104 via a vacuum tube 106 and an instillation tube 108. In the embodiments described herein, the dressing 104 is configured for use in treating one or more wounds on a patient's hand. The NPWTi system 100 is also shown to include an instillation fluid source 110 fluidly coupled to the instillation tube 108. The NPWTi system 100 is configured to provide negative pressure wound therapy at a wound bed by reducing the pressure at the dressing 104 relative to atmospheric pressure. The NPWTi system 100 is also configured to provide instillation therapy by providing instillation fluid to the dressing 104. By providing both negative pressure wound therapy and instillation therapy, the NPWTi system 100 is configured to facilitate wound healing. As described in detail below, the NPWTi system 100 is also configured to provide a physiotherapy mode that facilitates mobility, articulation, etc. of a patient's hand during treatment by the NPWTi system 100. The NPWTi system 100 thereby facilitates wound healing while also allowing for functional rehabilitation of the hand and reducing the risk of contractures.

Although the examples described herein show a NPWTi system 100 configured to provide both negative pressure wound therapy and instillation therapy, in other embodiments the system 100 is configured to provide negative pressure wound therapy (NPWT) without instillation therapy.

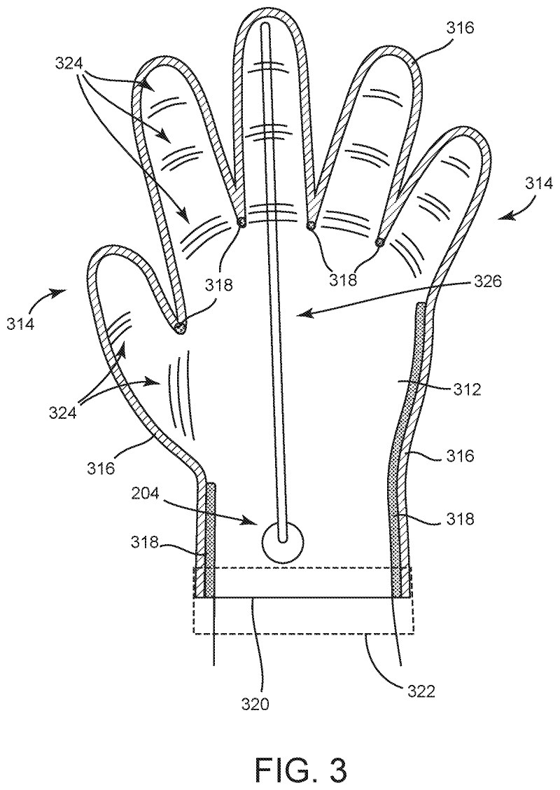

The dressing 104 is coupleable to a wound bed, i.e., a location of a wound (e.g., sore, laceration, burn, etc.) on a patient. In the examples herein, the dressing 104 is configured to be placed on a hand of a patient to cover a wound bed located on the hand. The dressing 104 may be substantially sealed over/around the wound bed such that a pressure differential may be maintained between the atmosphere and the wound bed (i.e., across the dressing 104). The dressing 104 may be coupled to the vacuum tube 106 and the instillation tube 108, for example to place the vacuum tube 106 and/or the instillation tube 108 in fluid communication with the wound bed. Embodiments of the dressing 104 are shown in FIGS. 3-9 and described in detail with reference thereto.

The dressing 104 includes one or more sensors 204. The one or more sensor(s) 204 are configured to measure one or more physical parameters at the dressing and provide the measurements to the control circuit 202, for example by transmitting the measurements via wireless communications (e.g., via a wireless network such as Bluetooth, WiFi, etc.). In the embodiments shown herein, the one or more sensor(s) 204 include a humidity sensor configured to measure humidity at the dressing 104, a moisture sensor configured to measure moisture at the dressing 104, and a strain sensor configured to measure a strain on the dressing 104. In some embodiments, the one or more sensor(s) 204 include one or more pH sensors to measure tissue pH or fluid pH.

The therapy unit 102 includes a negative pressure pump 112 (shown in FIG. 2 and obscured within the therapy unit 102 in the perspective view of FIG. 1) configured to pump air, wound exudate, and/or other debris (e.g., necrotic tissue) and/or fluids (e.g., instillation fluid) out of the dressing 104 via the vacuum tube 106, thereby creating a negative pressure at the dressing 104. The negative pressure pump 112 is fluidly communicable with the vacuum tube 106 and the dressing 104. Wound exudate and/or other debris and/or fluids removed from the wound bed by the negative pressure pump 112 may be collected in a canister 114 located on the therapy unit 102. The canister 114 may be removable from the therapy unit 102 to allow canister 114 to be emptied or replaced when the canister 114 fills with fluid and debris.

Operating the negative pressure pump 112 may therefore both create a negative pressure at the wound bed and remove undesirable fluid and debris from the wound bed. In some cases, operating the negative pressure pump 112 may cause deformation of the wound bed and/or provide other energy to the wound bed to facilitate debridement and healing of the wound bed. In various embodiments, the negative pressure pump 112 may be operated to provide various levels (amounts, values, etc.) of negative pressure at the wound bed (e.g., 30 mmHg, 60 mmHg, 75 mmHg, 125 mmHg, 150 mmHg, etc.) for example varying over time as part of a dynamic pressure control approach. In the embodiments described below, the negative pressure pump 112 is configured to operate, as controlled by the control circuit 202, to provide a first level of negative pressure at the wound bed corresponding to a wound therapy mode (e.g., 125 mmHg) and a second level of negative pressure at the wound bed corresponding to a physiotherapy mode (e.g., 60 mmHg), where the second level is closer to ambient air pressure than the first level.

The therapy unit 102 also includes an instillation pump 116. The instillation pump 116 is configured to selectively provide instillation fluid from the instillation fluid source 110 to the dressing 104. The instillation pump 116 is operable to control the timing and amount (volume) of instillation fluid provided to the dressing 104. The instillation pump 116 may be controlled in coordination with the negative pressure pump 112 to provide one or more wound treatment cycles that may facilitate wound healing. In some embodiments, the amount of fluid provided by the instillation pump is automatically determined using a wound volume estimation process executed by the therapy unit 102.

The therapy unit 102 is also shown to include an input/output device 118. The input/output device 118 is configured to provide information relating to the operation of the NPWTi system 100 to a user and to receive user input from the user. The input/output device 118 may display status information relating to the NPWTi system 100, for example including measurements obtained from the sensor(s) 204 of the dressing 104 or the sensor(s) 200 of the therapy unit 102. The input/output device 118 may allow a user to input various preferences, settings, commands, etc. that may be used in controlling the negative pressure pump 112 and the instillation pump 116 as described in detail below. The input/output device 118 may include a display (e.g., a touchscreen), one or more buttons, one or more speakers, and/or various other devices configured to provide information to a user and/or receive input from a user.

As shown in FIG. 2, the therapy unit 102 is also shown to include one or more sensors 200 and a control circuit 202. The sensor(s) 200 may be configured to monitor one or more of various physical parameters relating to the operation of the NPWTi system 100. For example, the sensor(s) 200 may measure pressure at the vacuum tube 106, which may be substantially equivalent and/or otherwise indicative of the pressure at the dressing 104. As another example, the sensor(s) 200 may measure an amount (e.g., volume) of instillation fluid provided to the dressing 104 by the instillation pump 116. The sensor(s) 200 may provide such measurements to the control circuit 202.

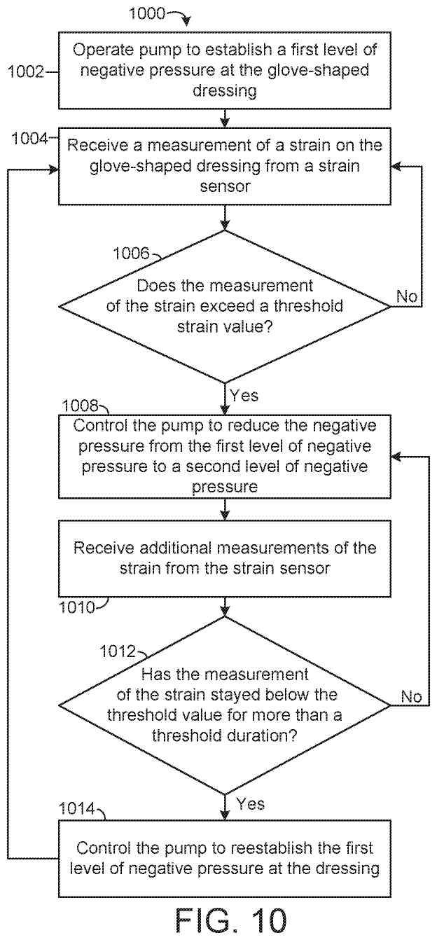

The control circuit 202 is configured to control the operation of the therapy unit 102, including by controlling the negative pressure pump 112, the instillation pump 116, and the input/output device 118. The control circuit 202 may receive measurements from the sensor(s) 200 and the sensor(s) 204 and/or user input from the input/output device 118 and use the measurements and/or the user input to generate control signals for the instillation pump 116 and/or the negative pressure pump 112. For example, the control circuit 202 may control the negative pressure pump 112 and the instillation pump 116 to provide various combinations of various instillation phases, soak periods, and negative pressure phases (i.e., various pressures and instillation amounts over various durations) to support and encourage wound healing. As another example, as described in detail below with reference to FIG. 10, the control circuit 202 is configured to automatically initiate a wound therapy mode in response to strain measurements from the sensor(s) 204 by controlling the negative pressure pump 112 to reduce the negative pressure at the dressing 104, thereby allowing increased mobility, flexion, articulation, etc. of the hand treated by the dressing 104.

### Hand Dressing for NPWTi or NPWT

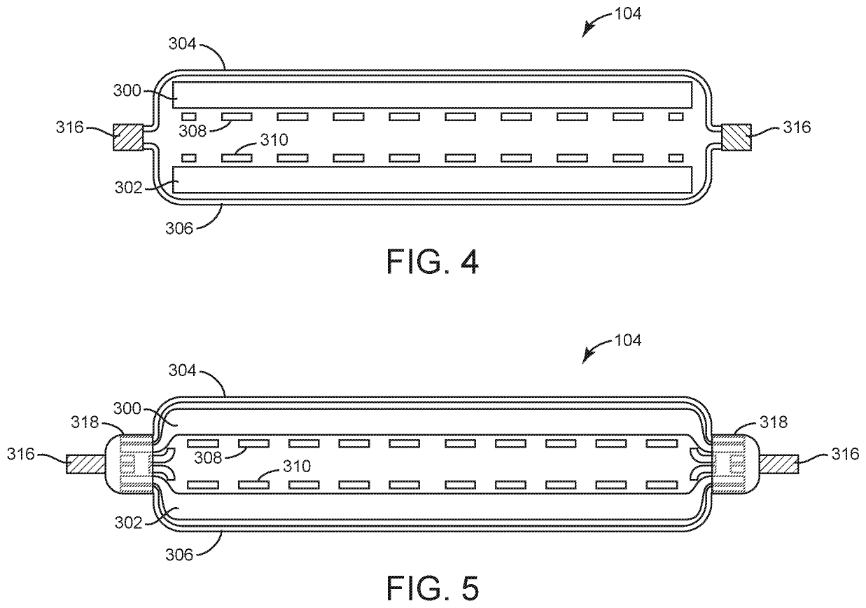

Referring now to FIGS. 3-5, various views of a first embodiment of the dressing 104 is shown. FIG. 3 shows a top view of the dressing 104 and FIGS. 4-6 show various cross-sectional views of the dressing 104.

In FIGS. 3-5, the dressing 104 is shown to include a first manifold layer 300, a second manifold layer 302, a first barrier layer 304 that is adjacent to (e.g., abuts) the first manifold layer 300, and a second barrier layer 306 abuts the second manifold layer 302. The first manifold layer 300 and the second manifold layer 302 are positioned between the first barrier layer 304 and the second barrier layer 306. In some embodiments, the first manifold layer 300 is coupled to the first barrier layer 304 by an adhesive and/or the second manifold layer 302 is coupled to the second barrier layer 306 by an adhesive. In other embodiments, the manifold layers 300, 302 are not adhered to the barrier layers 304, 306, thereby allowing the manifold layers 300, 302 to move, shift, etc. relative to the barrier layers 300, 302 to facilitate freedom of movement of a hand or other appendage within the dressing 104.

The dressing 104 is also shown to includes a first fenestrated film layer 308 that abuts the first manifold layer 300 with and a second fenestrated film layer 308 that abuts the second manifold layer 302. The first manifold layer 300 is positioned between the first fenestrated film layer 308 and the first barrier layer 304, and the second manifold layer 302 is positioned between the second fenestrated film layer 310 and the second barrier layer 306. In some embodiments, the first fenestrated film layer 308 is coupled to the first manifold layer 300 by an adhesive and/or the second fenestrated film layer 310 is coupled to the second manifold layer 302 by an adhesive. In preferred embodiments. The first fenestrated film layer 308 is configured to be easily separated from the second fenestrated film layer 310. That is, the first fenestrated film layer 308 and the second fenestrated film layer 310 are configured to not adhere to one another.

As illustrated in FIG. 5, the first manifold layer 300, the second manifold layer 302, the first barrier layer 304, the second barrier layer 306, the first fenestrated film layer 308, and the second fenestrated film layer 310 are hand-shaped. That is, each of the layers 302-310 includes a central region 312 and five peninsular projections 314 that extend from the central region 312 in the shape of a hand. Each of the five peninsular projections 314 corresponds to one finger or thumb of a patient. The dressing 104 may be made available in various sizes corresponding to different hand sizes (i.e., different dimensions of the central region 312 and the peninsular projections 314 of the layers 300-310). For example, the dressing 104 may be available in a small size, a medium size, a large size, etc. to allow fitting to various patients without requiring individual/patient-specific customization.

The first barrier layer 304 is coupled to the second barrier layer 306 along a hand portion of a perimeter of the dressing 104 and separated from the second barrier layer 306 along a wrist portion 320 of the perimeter of the dressing 104. The first barrier layer 304 is not coupled to the second barrier layer 306 along the wrist portion 320 of the perimeter of the dressing 104, which creates an opening that allows a patient's hand to be inserted into the dressing 104. In other words, the dressing 104 is formed as a glove. The dressing 104 is thereby configured to receive a patient's hand between the first fenestrated film layer 308 and the second fenestrated film layer 310.

In the example shown, the first barrier layer 304 is coupled to the second barrier layer 306 along edges of the peninsular regions 314 and the central region 312 by film welds 316, and along a portion of the perimeter of the central region by anchor welds 318. Anchor welds 318 are also shown as located at points joining the peninsular regions 314. FIG. 4 shows a cross-section view of the dressing 104 including film welds 316. The film welds 316 couple the first barrier layer 304 to the second barrier layer 306 and substantially prevent air from passing between the first barrier layer 304 and the second barrier layer 306 at the film welds. For example, the first barrier layer 304 may be thermally bonded to the second barrier layer 306 at the film welds 316. As another example, the film welds 316 may be formed by adhesive bonds (e.g., using acrylic adhesives).

In other example embodiments, the anchor welds 318 are omitted such that the barrier layers 304, 306 enclose the manifold layers 300, 302 and the film layers 308, 310 while allowing the manifold layers 300, 302 and the film layers 308, 310 to move substantially freely relative to the barrier layers 304, 306. In some such embodiments, the manifold layers 300, 302 and the film layers 308, 310 are coupled together. In yet other example embodiments, all six layers (i.e., the first manifold layer 300, the second manifold layer 302, the first barrier layer 304, the second barrier layer 306, the first film layer 308, and the second film layer 310) are all thermally bonded together around a periphery of the dressing 104, except along the wrist portion 320.

FIG. 5 shows a cross-section view of the dressing includes film welds 316 and anchor welds 318. The anchor welds 318 couple the first manifold layer, the second manifold layer 302, the first barrier layer 304, the second barrier layer 306, the first fenestrated film layer 308, and the second fenestrated film layer 310 together along portions of the perimeter of the dressing where the anchor welds 318 are present. In the example shown, the anchor welds 318 include structures (e.g., staples, pins, etc.) extending through the layers 300-310 to restrict (e.g., substantially prevent) movement of the layers 300-310 relative to one another at the anchor welds 318. In other examples, adhesive is used along the anchor welds 318 to restrict movement of the layers 300-310 relative to one another at the anchor welds 318.

The dressing 104 is also shown to include an adhesive cuff 322. Adhesive cuff 322 includes an adhesive (or multiple adhesives) configured to seal the adhesive cuff 322 to the first barrier layer 304 and the second barrier layer 306 along the wrist portion 320 of the perimeter of the dressing and to skin of a patient. The adhesive cuff 322 extends from the first barrier layer 304 and the second barrier layer 306 such that the adhesive cuff 322 is configured to be coupled to a wrist of a patient when the patient's hand is inserted into the dressing 104. When the adhesive cuff 322 is sealed to a patient's wrist, the first barrier layer 304, and the second barrier layer 306, the adhesive cuff 322 substantially prevents air from flowing between an ambient environment and the interior of dressing 104 (e.g., the manifold layers 300, 302) via the opening at the wrist portion 320 of the dressing 104. The adhesive cuff 322 may be produced as an integrated piece of the dressing 104 or may be distributed as a separate piece of a dressing kit (e.g., as an adhesive strip).

The barrier layers 304, 306 are configured to substantially prevent airflow therethrough. The barrier layers 304, 306 may include a polyurethane drape material, for example a drape material as used in a V.A.C.® Drape by Acelity. As mentioned above, the barrier layers 304, 306 are sealed with a substantially-airtight seal by film welds 316. Accordingly, when the adhesive cuff 322 is sealed around the wrist of a patient and the barrier layers 304, 306, a substantially airtight volume is created within the dressing 104, i.e., between the barrier layers 304, 306 and the patient's hand. The barrier layers 304, 306 may each have a thickness in a range between approximately 80 and 120 microns.

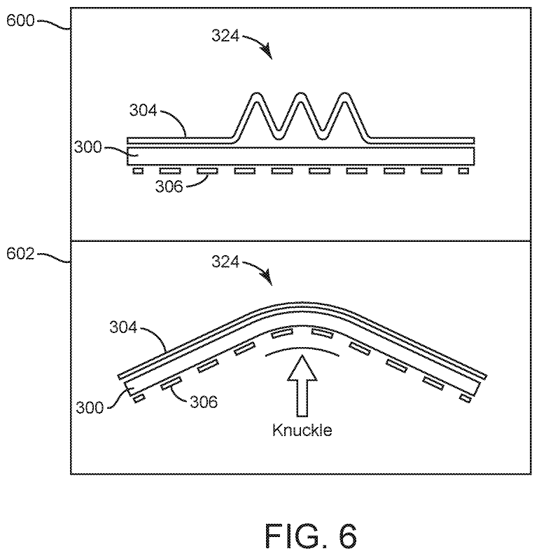

As shown in FIG. 3, the first barrier layer 304 includes knuckle flexion points 324 arranged at positions that correspond to knuckles/joints within a typical hand that may be inserted into the dressing 104. In the example shown, each peninsular portion 314 corresponding to a finger includes three knuckle flexion points 324, while the peninsular portion 314 corresponding to a thumb includes two knuckle flexion points. FIG. 6 shows cross sectional views of a knuckle flexion point 324, includes a first view 600 of the knuckle flexion point 324 in an unflexed state and a second view 602 of the knuckle flexion point 324. As illustrated by FIG. 6, each knuckle flexion point 324 includes a series of folds (e.g., three folds) which, in the unflexed state, draw the barrier layer 304 away from the manifold layer 300. In the flexed state, the series of folds are extended (unfolded) to facilitate curvature (bending) of the dressing 104 at the knuckle flexion point 324 by increasing an effective length of the barrier layer 304. Accordingly, the knuckle flexion points 324 are configured to facilitate articulation, movement, etc. of a patient's fingers confined in the dressing 104. The knuckle flexion points 324 may be formed by thermoforming. The fenestrated film layers 308, 310 and the manifolding film layers 300, 302 may be configured to resiliently stretch and/or flex to accommodate articulation, movement, etc. of a hand in the dressing 104 as shown in FIG. 6.

The fenestrated film layers 308, 310 are made of a non-adherent film and are configured to provide a non-adherent interface between the dressing 104 and a hand of a patient, including a wound bed located on the hand. The fenestrated film layers 308, 310 are also configured to prevent ingrowth of skin to the dressing (e.g., healing into the manifold layers 300, 302). The fenestrated film layer 308, 310 thereby facilitate easy insertion of a hand into the dressing 104 and removal of the hand from the dressing 104. Additionally, the fenestrated film layers 308, 310 have fenestrations (perforations, holes, airways, windows, etc.) extending therethrough that allow air and fluid to pass between the hand (e.g., a wound bed) and the manifold layers 300, 302. The fenestrations may have a length in a range between approximately 2 millimeters and approximately 5 millimeters (e.g., approximately 3 millimeters) and a width of less equal to or less than approximately 0.5 millimeters. A spacing between the fenestrations may be approximately equal to the length of the fenestrations (e.g., approximately 3 millimeters). In some embodiments, the fenestrations are arranged in staggered rows, such that a fenestration in a first row aligns with a gap between fenestrations in a neighboring row. In other embodiments, the fenestrations are aligned in rows and columns, are aligned to various angles relative to one another, are aligned to alternating right-angle orientations, or are arranged in some other pattern. The fenestrations may be elastic passages which expand or open in response to a pressure gradient across the film layers 308, 310, and at least partially close to restrict fluid flow therethrough in the absence of a pressure gradient across the film layers 308, 310. In some embodiments, each fenestrated film layers 308, 310 may thereby form a seal in the absence of a pressure gradient. The fenestrated film layers 308, 310 may be made of a polymeric film and may be hydrophobic. The fenestrated film layers 308, 310 may each have a thickness of approximately 30 microns.

The manifold layers 300, 302 are configured to allow air and fluid to flow therethrough. The manifold layers are made of an open-cell foam, for example a reticulated polyurethane open cell foam. In some embodiments, the manifold layers 300, 302 are made of an open-cell foam marketed as GRANUFOAM™ by ACELITY™. The manifold layers 300, 302 may each have a thickness in a range between approximately 6 mm and 10 mm. Accordingly, the manifold layers 300, 302 may be thinner than in conventional bulky dressings. The reduced thickness of the manifold layers 300, 302 facilitates flexion of the dressing 104 to allow for physiotherapy for the hand in the dressing 104 in a way not previously achieved.

The manifold layers 300, 302 allow for the communication of air pressure, for example negative pressure (relative to ambient air pressure), through the manifold layers 300, 302 and to the hand and the wound bed (via the fenestrated film layers 308, 310. The dressing 104 is configured such that air and fluid can flow between the first manifold layer 300 and the second manifold layer 302 proximate the film welds 316 and anchor welds 318, i.e., through the fenestrated film layers 308, 310 and around a hand positioned in the dressing 104. Negative pressure can thereby be communicated across both manifold layers 300, 302 (i.e., such that both manifold layers 300, 302 are maintained at approximately equal pressures).

The dressing 104 is configured to be coupled to a vacuum (negative pressure) tube 106 and, in some embodiments, an instillation tube 108. For example, a hole may be cut in the first barrier layer 304 (e.g., with a diameter in a range between approximately 3-20 mm) and a connection pad may be coupled to the barrier layer 304 over the hole. The connection pad is coupled to the vacuum tube 106 and/or instillation tube 108. In some embodiments, multiple holes and/or connection pads are used. For example, the connection pad may be a SENSAT.R.A.C.™ connection pad marketed by ACELITY™.

The manifold layers 300, 302 can thereby be put in fluid communication with the vacuum tube 106 and/or instillation tube 108. As described above with reference to FIGS. 1-2, the negative pressure pump 112 can be controlled to remove air from the manifold layers 300, 302 to establish a negative pressure at the manifold layers 300, 302. The negative pressure at the manifold layers 300, 302 is communicated to the hand/wound via the fenestrations in the fenestrated film layers 308, 310. Instillation fluid may also be provided to the wound via the manifold layers 300, 302 and the fenestrated film layers 308. Wound exudate, instillation fluid, other debris, etc. may also be removed from the wound and manifold layers via the vacuum tube 106 as described above with reference to FIGS. 1-2. The dressing 104 thereby facilitates treatment of a hand wound using NPWTi.

Still referring to FIGS. 3-6, the dressing 104 is also shown to include one or more sensor(s) 204 positioned on the first barrier layer 304. In the embodiment shown, the one or more sensor(s) include a humidity sensor and a moisture sensor, which may be positioned extending through the first barrier layer 304 to measure humidity and moisture in the first manifold layer 300. In some embodiments, the one or more sensor(s) include one or more pH sensor(s) configured to measure tissue pH and/or fluid pH. In the embodiment shown, the one or more sensor(s) also include a strain sensor 326. The strain sensor 326 is positioned on or in the first barrier layer 304 and extends along a length of the dressing from proximate the wrist portion 320 to a tip of one of the peninsular regions 314 (e.g., corresponding to a middle finger). The strain sensor 326 is configured to measure (e.g., generate an electrical signal indicative of) a strain on the dressing 104 (i.e., on the strain sensor 326), which may correspond to a curvature of the dressing 104 and/or a force applied by the hand inside the dressing 104. For example, a strain measured by the strain sensor 326 may increase when a patient attempts to clench the hand (e.g., in a first) or otherwise bend one or more fingers in the dressing 104. The strain may decrease when the patient moves the hand in the dressing 104 to an open or neutral pose. The one or more sensors 204 include a wireless communications circuit (e.g., WiFi transceiver, Bluetooth transceiver, etc.) configured to facilitate wireless transmission of measurements from the one or more sensors to the control circuit 202 of the therapy unit 102.

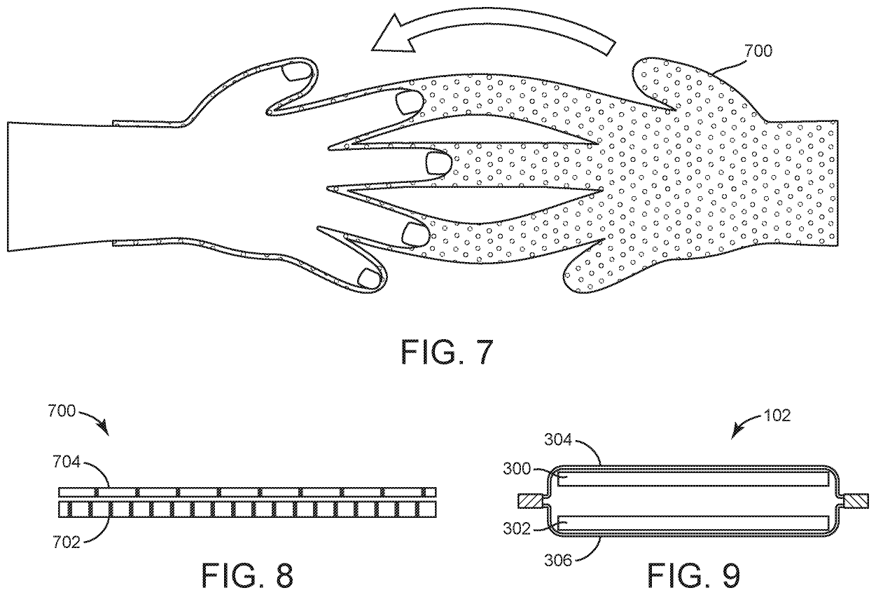

Referring now to FIGS. 7-9, a second embodiment of the dressing 104 is shown, according to an exemplary embodiment. In FIGS. 7-9, the non-adhesive fenestrated film layers 308, 310 are omitted from the dressing 104, such that the dressing 104 is formed as a glove including the barrier layers 304, 306 and the manifold layers 300, 302 arranged as described above. A wound-dressing interface 700 is also included as a separate piece (i.e., distributed to caregivers/patients as a separate piece in a dressing kit that also includes the glove-shaped dressing 104 formed from the barrier layers 304, 306 and the manifold layers 300, 302). The wound-dressing interface 700 is formed as a single piece (sheet) as shown in FIG. 4, for example shaped within peninsular extensions and or bridge/isthmus-shaped portions configured to be aligned with fingers of a patient when the wound-dressing interface 700 is folded over a patient's hand.

The wound-dressing interface 700 includes a patient interface layer 702 and a foam interface layer 704. The foam interface layer 704 includes a fenestrated film, for example a polyurethane or polyethylene film with fenestrations extending therethrough. The foam interface layer 704 allows air and fluid to flow therethrough and limits adherence of the wound-dressing interface 700 to the manifold layers 300, 302. The patient interface layer 702 includes a perforated silicone and a hydrogel or polyurethane gel. The patient interface layer 702 is configured to adhere to itself. In some embodiments, the patient interface layer 702 is configured to be of low tackiness against the skin or wound, particularly when wet. In some embodiments, the patient interface layer 702 is omitted.

The wound-dressing interface 700 is thereby configured to be folded over a hand and adhered to itself (mated to itself) to substantially enclose the hand in the wound dressing interface 700 such that the patient interface layer 702 faces inwards (i.e., towards the hand) and the foam interface layer 704 faces outwards (i.e., away from the hand). The hand and the wound-dressing interface 700 can then be inserted into the glove portion of the dressing 104, i.e., the barrier layers 304, 306 and the manifold layers 300, 302 arranged as described above (and as shown in FIGS. 9 and 3). With the hand enclosed in the wound-dressing interface 700, the wound-dressing interface 700 prevents direct contact between the hand and the manifold layers 300, 302 while allowing air and fluid to pass through fenestrations in the wound-dressing interface 700. The adhesive cuff 322 can then be applied around the patient's wrist to seal the dressing 104 around the hand as described above. To further prepare the dressing 104 for NPWTi, a hole can be cut in a barrier layer 304, 306 and a connection pad coupled to the barrier layer 304, 306 over the hole to place a vacuum tube 106 and/or an instillation tube 108 in fluid communication with the manifold layers 300, 302. The therapy unit 102 can then be operated as described above to establish negative pressure at the hand and/or provide instillation fluid to the hand.

The embodiments of FIGS. 3-9 show glove-shaped dressings, i.e., with individually-differentiated fingers (e.g., as formed by peninsular projections 314). Other embodiments of the dressing 104 may be mitten-shaped, i.e., with a unified area for four fingers and a separate projection for a thumb. Such mitten-shaped dressings may otherwise be configured as described herein for the glove-shaped dressings of FIGS. 3-9. Other variations are also contemplated by the presented disclosure, for example a three-compartment glove where the two pairs of fingers each share a compartment and the thumb has a compartment, etc. All such variations are within the scope of the present disclosure.

Referring now to FIG. 10, a process 1000 of providing a physiotherapy mode with the NPWTi system 100 of FIGS. 1-2 and the hand dressing 104 of FIGS. 3-9 is shown, according to an exemplary embodiment. Process 1000 provides a physiotherapy mode that allows movement, articulation, bending, etc. of a hand in the dressing 104 during NPWTi treatment. Accordingly, execution of process 1000 facilitates a patient in redeveloping strength, neuromuscular activity, coordination, etc. in the hand while the dressing 104 is applied to the hand. Additionally, movement of the hand as provided for by process 1000 reduces the risk of contracture, i.e., the risk that the skin may heal too tight such that the patient's skin restricts the range of motion of the joints in the hand. Movement, articulation, etc. of the fingers and hand during wound healing may facilitate proper healing that allows for a full range of motion of the hand after wound healing. Process 1000 can be executed by the control circuit 202 of the therapy unit 102.

At step 1002, the negative pressure pump 112 is operated to establish a first level of negative pressure at the glove-shaped dressing 104. The first level of negative pressure may correspond to a preferred level for negative pressure wound therapy, for example in the range of approximately 100 mmHg to 175 mmHg of negative pressure. When the first level of negative pressure is applied, the pressure differential between the ambient air and the interior of the dressing 104 increases the rigidity of the dressing 104 such that dressing 104 substantially restricts (limits, prevents, etc.) articulation of the hand.

At step 1004, a measurement is received from the strain sensor 326 on the glove-shaped dressing 104. The measurement includes a current value of a strain on the dressing 104. The strain on the dressing 104 may correspond to an amount of force exerted on the dressing 104 by the hand in the dressing 104 in an attempt to curl, bend, articulate, etc. the fingers in the dressing 104. The measurement may be received by the control circuit 202 via a wireless network (e.g., Bluetooth communications, WiFi communications, etc.).

At step 1006, the measurement is compared to a threshold strain value. The threshold strain value may be predetermined, for example by bench testing. The threshold strain value corresponds to a significant probability that the patient is deliberately attempting to articulate the hand in the dressing 104. In the measurement does not exceed the threshold measurement, pump 112 continues to be controlled to provide the first level of negative pressure at the dressing 104 while more measurements of the strain are received at the control circuit 202 over time.

If a determination is made that the measurement of the strain exceeds the threshold strain value, a physiotherapy mode is initiated at step 1008. At step 1008, the pump 112 is controlled (e.g., by the control circuit 202) to reduce the negative pressure from the first level of negative pressure to a second level of negative pressure. The second level of negative pressure is “lower” than the first level of negative pressure, i.e., closer to atmospheric pressure (e.g., in a range of approximately 25 mmHg to 75 mmHg). At the second level of negative pressure, the rigidity of the dressing 104 is lower than at the first level of negative pressure. Accordingly, at the second level of negative pressure, the dressing 104 and the NPWIT system 100 allows the patient to at least partially bend, articulate, move, etc. the fingers and hand in the dressing 104. For example, the patient may follow guided instructions from a therapist. In some embodiments, the therapy unit is configured to provide instructions for a physiotherapy routine to a user via the input/output device 118.

At step 1010, additional measurements of the strain are received from the strain sensor 234. As the patient continues to articulate the hand in the dressing 104, the strain will stay above the threshold strain value and/or repeatedly exceed the threshold strain value. At step 1012, a determination is made of whether the measurement has fallen below the threshold strain value for at least a threshold duration of time. The threshold duration of time may be selected as indicative that the patient has ended a physiotherapy routine or other attempt to articulate the hand in the dressing 104. If the strain has not fallen below the threshold strain value for at least the threshold duration of time, the pump 112 continues to be controlled to maintain the second level of negative pressure at the dressing.

If the strain has fallen below the threshold strain value for at least the threshold duration of time, the pump 112 is controlled to reestablish the first level of negative pressure at the dressing at step 1014, i.e., to reestablish an optimal NPWTi regime and exit the physiotherapy mode. The process may then return to step 1004 where the strain measurements are monitored. Repeated iterations of the physiotherapy mode may thereby be initiated and exited to facilitate both physiotherapy and NPWTi for the hand in the dressing 104 over time. With the advantages described above, the dressing 104 may be well-suited for long-term application to the hand (e.g., seven days or longer).

Several variations on the process 1000 are also contemplated by the present disclosure. For example, in some embodiments, the physiotherapy mode can be initiated or ended in response to user input to the input/output device 118 commanding a start or end to the physiotherapy mode. As another example, the control circuit 202 may prevent execution of the process 1000 (e.g., prevent initiation of physiotherapy mode) during an instillation cycle (e.g., while instillation fluid is being supplied to the dressing 104). As another example, in some embodiments, a dynamic pressure control mode (e.g., cyclic variations in negative pressure) is applied outside of the physiotherapy mode (e.g., in place of the first level of negative pressure). Various such variations are possible.

Additionally, although the embodiments described herein are designed for use on hands, variations suitable for use on feet or amputation stumps are also within the scope of the present disclosure. For example, a variation suitable for use on a foot may be formed as a sock, with or without a separate pocket/projection for each toe, rather than as a glove as shown for the hand dressings described above. Variations of the dressing 104 can therefore be tailored for use in treating wounds in many anatomical locations.

The dressing 104 and NPWTi system 100 described above provide various advantages over existing dressings and wound therapy systems. The dressing 104 is easy to apply (thereby reducing application time) and remove without damaging the healed/healing wound (e.g., by avoiding a risk of in-growth into the dressing structure). The dressing 104 and NPWTi system 100 also allow for effective positioning of the dressing 104 while also allowing early movement in the full range of motion (or at least a significant portion of the range of motion) of the wounded/treated hand. The dressing 104 and NPWTi system 100, in the embodiments shown, are suitable for providing negative pressure and instillation therapy for up to at least seven days. The dressing 104 may reduce the use of foam relative to existing dressings, thereby making the dressing 104 smaller and less cumbersome for the patient. The dressing 104 and the NPWTi system 100, in the embodiments shown, also provide for an automatic physiotherapy mode that facilitates rehabilitation and reduces the risk of contractures. Additionally, the dressing 104 includes sensors that wirelessly (e.g., without the annoyance/complication of additional cables/wires/etc.) communicate useful measurements/diagnostics to a caregiver that allow early detection of infection or other developments in wound treatment. Therefore, the dressing 104 and NPWTi system 100 disclosed herein provide many advantages over existing systems that can improve outcomes for patients while also improving the overall treatment experience.

### CONFIGURATION OF EXEMPLARY EMBODIMENTS

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, calculation steps, processing steps, comparison steps, and decision steps.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (IC), discrete circuits, system on a chip (SOCs) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

The “circuit” may also include one or more processors communicably coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations. The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

1. A dressing, comprising:
a first manifold layer;
a second manifold layer;
a first barrier layer coupled to the first manifold layer; and
a second barrier layer coupled to the second manifold layer;
a first fenestrated film layer coupled to the first manifold layer; and
a second fenestrated film layer coupled to the second manifold layer;
wherein the first manifold layer and the second manifold layer are positioned between the first barrier layer and the second barrier layer;
wherein the first fenestrated film layer and the second fenestrated film layer are positioned between the first manifold layer and the second manifold layer.

2. The dressing of claim 1, comprising: wherein the first manifold layer, the second manifold layer, the first barrier layer, and the second barrier layer are hand-shaped
wherein the first barrier layer is coupled to the second barrier layer along a hand portion of a perimeter of the dressing and separated from second barrier layer along a wrist portion of the perimeter of the dressing.

3. (canceled)

4. The dressing of claim 2, wherein the dressing is configured to receive a hand of a patient between the first fenestrated film layer and the second fenestrated film layer.

5. The dressing of claim 4, wherein the dressing comprises an adhesive cuff positioned at the wrist portion of the perimeter of the dressing, the adhesive cuff configured to be coupled to a wrist of a patient.

6. The dressing of claim 5, wherein the first barrier layer and the second barrier layer are substantially impermeable to air and wherein the adhesive cuff is configured to provide a substantially airtight seal between the first and second barrier layers and the wrist of the patient.

7. The dressing of claim 1, a connection pad coupled to the first barrier layer and configured to couple the dressing to a tube coupled to a pump such that the pump is in pneumatic communication with the first manifold layer and the second manifold layer.

8. The dressing of claim 7, wherein:
the pump is controllable to selectively establish a first level of negative pressure at the dressing and a second level of negative pressure at the dressing; and
the dressing is configured to substantially prevent bending of the dressing when the first level of negative pressure is established at the dressing and to allow bending of the dressing when the second level of negative pressure is established at the dressing.

9. (canceled)

10. The dressing of claim 1, comprising one or more sensors coupled to the dressing, the one or more sensors comprising one or more of a moisture sensor, a humidity sensor, a pH sensor, and a strain sensor.

11. (canceled)

12. The dressing of claim 1, wherein the first barrier layer comprises a plurality of knuckle flexion points, each knuckle flexion point configured to facilitate bending of the first barrier layer at the knuckle flexion point.

13. (canceled)

14. The dressing of claim 1, wherein the first fenestrated film layer is positioned between the first manifold layer and tissue of a patient when the dressing is applied to the patient.

15. The dressing of claim 1, wherein, when the dressing is applied to a patient, the first fenestrated film layer prevents the first manifold layer from contacting a tissue of the patient and the second fenestrated film layer prevents the second manifold layer from contacting the tissue of the patient.

16. (canceled)

17. (canceled)

18. (canceled)

19. (canceled)

20. (canceled)

21. (canceled)

22. A negative pressure wound therapy system, comprising:
a glove-shaped dressing;
a sensor coupled to the glove-shaped dressing;
a tube coupled to the glove-shaped dressing;
a pump coupled to the tube; and
a controller configured to receive a measurement from the sensor.

23. The negative pressure wound therapy system of claim 22, wherein the sensor is configured to collect a measurement of a strain on the glove-shaped dressing.

24. (canceled)

25. The negative pressure wound therapy system of claim 22, wherein the glove-shaped dressing is configured to be sealed around a hand of a patient;
wherein the glove-shaped dressing restricts bending of the hand when a first level of negative pressure is established at the glove-shaped dressing by the pump.

26. The negative pressure wound therapy system of claim 25, wherein the controller is configured to:
determine whether the measurement from the sensor exceeds a threshold value;
in response to a determination that the measurement from the sensor exceeds a threshold value, control the pump to provide a second level of negative pressure at the glove-shaped dressing, the second level closer to ambient air pressure than the first level.

27. The negative pressure wound therapy system of claim 26, wherein the glove-shaped dressing allows bending of the hand when the second level of negative pressure is established at the glove-shaped dressing by the pump.

28. (canceled)

29. (canceled)

30. The negative pressure wound therapy system of claim 29, wherein the sensor extends from a wrist region of the glove-shaped dressing to a fingertip region of the glove-shaped dressing.

31. The negative pressure wound therapy system of claim 22, wherein the sensor comprises at least one of a humidity sensor or a moisture sensor, wherein the controller is configured to receive measurements from the at least one of the humidity sensor or the moisture sensor via a wireless network.

32. A method for controlling a pump coupled to a glove-shaped dressing, the method comprising:
controlling the pump to establish a first level of negative pressure at the glove-shaped dressing;
receiving a measurement of a strain on the glove-shaped dressing from a strain sensor coupled to the glove-shaped dressing;
determining whether the measurement of the strain exceeds a threshold value;
in response to a determination that the measurement of the strain exceeds the threshold value, initiating a physiotherapy mode by controlling the pump to establish a second level of negative pressure at the glove-shaped dressing, the second level of negative pressure closer to ambient air pressure that the first level of negative pressure.

33. The method of claim 32, comprising:
determining that the measurement of the strain on the glove-shaped dressing is below the threshold value for more than a threshold duration; and
in response to a determination that the measurement of the strain on the glove-shaped dressing returned below the threshold value for more than the threshold duration, exiting the physiotherapy mode by controlling the pump to establish the first level of negative pressure at the glove-shaped dressing.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)
